SEQUENCE LISTING (1 sequence)

SEQ ID NO: 1 - DNA, 22 bp, Artificial Sequence
  source: /note=“Description of Artificial Sequence Synthetic oligonucleotide”
caatgccatcctggagttcctg

(19) United States
(12) Patent Application Publication
(10) Pub. No.: US 2022/0152086 A1
(43) Pub. Date: May 19, 2022

(54) METHODS FOR TREATING MUSCULAR DYSTROPHY WITH CASIMERSEN

(71) Applicant: Sarepta Therapeutics, Inc., Cambridge, MA (US)

(72) Inventor: Edward M. Kaye, Cambridge, MA (US)

(21) Appl. No.: 17/441,620

(22) PCT Filed: Mar. 25, 2020
(86) PCT No.: PCT/US2020/024550

§ 371 Date: Sep. 21, 2021

Related U.S. Application Data
(60) Provisional application No. 62/825,573, filed on Mar. 28, 2019.
(60) Provisional application No. 62/902,518, filed on Sep. 19, 2019.

(51) Int. Cl. A61K 31/7125; A61P 21/00

(52) U.S. Cl. CPC A61K 31/7125; A61P 21/00

(57) ABSTRACT

The present disclosure provides, among other things, improved compositions and methods for treating muscular dystrophy. For example, the disclosure provides methods for treating Duchenne muscular dystrophy patients having a mutation in the DMD gene that is amenable to exon 45 skipping by administering an effective amount of casimersen.

### RELATED APPLICATIONS

This application claims the benefit of U.S. Provisional Application No. 62/825,573, filed on Mar. 28, 2019 and U.S. Provisional Application No. 62/902,518, filed on Sep. 19, 2019. The entire teachings of the above-referenced applications are incorporated by reference in their entirety.

### FIELD OF THE INVENTION

The present invention relates to improved methods for treating muscular dystrophy in a patient. It also provides compositions suitable for facilitating exon 45 skipping in the human dystrophin gene.

### BACKGROUND OF THE INVENTION

In a variety of genetic diseases, the effects of mutations on the eventual expression of a gene can be modulated through a process of targeted exon skipping during the splicing process. In cases where a normally functional protein is prematurely terminated because of mutations therein, a means for restoring some functional protein production through antisense technology has been shown to be possible through intervention during the splicing processes, and that if exons associated with disease-causing mutations can be specifically deleted from some genes, a shortened protein product can sometimes be produced that has similar biological properties of the native protein or has sufficient biological activity to ameliorate the disease caused by mutations associated with the exon (see e.g., Sierakowska, Sambade et al. 1996; Wilton, Lloyd et al. 1999; van Deutekom, Bremmer-Bout et al. 2001; Lu, Mann et al. 2003; Aartsma-Rus, Janson et al. 2004).

Duchenne muscular dystrophy (DMD) is caused by a defect in the expression of the protein dystrophin. Dystrophin is a rod-shaped cytoplasmic protein, and a vital part of a protein complex that connects the cytoskeleton of a muscle fiber to the surrounding extracellular matrix through the cell membrane. Dystrophin plays an important structural role in the muscle fiber, connecting the extracellular matrix and the cytoskeleton. The N-terminal region binds actin, whereas the C-terminal end is part of the dystrophin glycoprotein complex (DGC), which spans the sarcolemma. It has been shown that dystrophin-deficient muscle fibers of the mdx mouse exhibit an increased susceptibility to contraction-induced sarcolemma rupture (see Petrof et al. 1993; Cirak et al. 2012).

The gene encoding the dystrophin protein contains 79 exons spread out over more than 2 million nucleotides of DNA. Any exonic mutation that changes the reading frame of the exon, or introduces a stop codon, or is characterized by removal of an entire out of frame exon or exons, or duplications of one or more exons, has the potential to disrupt production of functional dystrophin, resulting in DMD.

Disease onset can be documented at birth with elevated creatine kinase levels, and significant motor deficits may be present in the first year of life. By the age of seven or eight, most patients with DMD have an increasingly labored gait and are losing the ability to rise from the floor and climb stairs; by ages 10 to 14, most are wheelchair-dependent. DMD is uniformly fatal; affected individuals typically die of respiratory and/or cardiac failure in their late teens or early 20s. The continuous progression of DMD allows for therapeutic intervention at all stages of the disease; however, until recently treatment was limited to glucocorticoids, which are associated with numerous side effects including weight gain, behavioral changes, pubertal changes, osteoporosis, Cushingoid facies, growth inhibition, and cataracts. Consequently, developing better therapies to treat the underlying cause of this disease is imperative.

A less severe form of muscular dystrophy, Becker muscular dystrophy (BMD) has been found to arise where a mutation, typically a deletion of one or more exons, results in a correct reading frame along the entire dystrophin transcript, such that translation of mRNA into protein is not prematurely terminated. If the joining of the upstream and downstream exons in the processing of a mutated dystrophin pre-mRNA maintains the correct reading frame of the gene, the result is an mRNA coding for a protein with a short internal deletion that retains some activity, resulting in a Becker phenotype.

For many years it has been known that deletions of an exon or exons which do not alter the reading frame of a dystrophin protein would give rise to a BMD phenotype, whereas an exon deletion that causes a frame-shift will give rise to DMD (Monaco, Bertelson et al. 1988). In general, dystrophin mutations including point mutations and exon deletions that change the reading frame and thus interrupt proper protein translation result in DMD. It should also be noted that some BMD and DMD patients have exon deletions covering multiple exons.

Recent clinical trials testing the safety and efficacy of splice switching oligonucleotides (SSOs) for the treatment of DMD are based on SSO technology to induce alternative splicing of pre-mRNAs by steric blockade of the spliceosome (Cirak et al., 2011; Goemans et al., 2011; Kinali et al., 2009; van Deutekom et al., 2007). However, despite these successes, the pharmacological options available for treating DMD are limited.

Thus, there remains a need for improved compositions and methods for producing dystrophin and treating muscular dystrophy, such as DMD and BMD in patients.

### SUMMARY OF THE INVENTION

The present disclosure is based, at least in part, on clinical evidence showing that treatment with an exon 45 skipping antisense oligonucleotide, casimersen, significantly increased dystrophin protein in patients over baseline. Furthermore, a positive correlation between exon skipping and de novo dystrophin protein was observed.

Accordingly, in some aspects, the disclosure provides a method for treating Duchenne muscular dystrophy (DMD) in a patient in need thereof who has a mutation of the DMD gene that is amenable to exon 45 skipping, comprising administering to the patient a dose of casimersen or a pharmaceutically acceptable salt thereof.

In some aspects, the disclosure provides, a method for restoring an mRNA reading frame to induce exon skipping in a patient with Duchenne muscular dystrophy (DMD) in need thereof who has a mutation of the DMD gene that is amenable to exon 45 skipping, comprising administering to the patient a dose of casimersen or a pharmaceutically acceptable salt thereof.

In some aspects, the disclosure provides, a method for increasing dystrophin production in a patient with Duchenne muscular dystrophy (DMD) in need thereof who has a mutation of the DMD gene that is amenable to exon 45 skipping, comprising administering to the patient a dose of casimersen or a pharmaceutically acceptable salt thereof.

In some aspects, the dose is administered at a dosage of about 4 mg/kg, about 10 mg/kg, about 20 mg/kg, about 30 mg/kg, about 40 mg/kg, or about 50 mg/kg of body weight of the patient.

In some aspects, the dose is administered as a single dose. In some aspects, the dose is administered once weekly. In some aspects, the dose is administered intravenously. In some aspects, the dose is administered intravenously by infusion. In some aspects, the dose is administered intravenously by infusion over a period of 35-60 minutes. In some aspects, the dose is administered intravenously by subcutaneous injection.

In some aspects, the patient is up to 40 years old, up to 30 years old, or up to 21 years old. In some aspects, the patient is 1 to 21 years old. In some aspects, the patient is 5 to 21 years old. In some aspects, the patient is 7 to 13 years old.

In some aspects, the disclosure provides a method according to any of the foregoing or related aspects, wherein the patient has a mutation of the DMD gene that is selected from the group including exons 7 to 42, 12 to 42, 18 to 42, 44 to 46, 44 to 47, 44 to 48, 44 to 49, 44 to 51, 44 to 53, 44 to 55, 44 to 57, or 44 to 59, or exon 44.

In some aspects, the disclosure provides methods according to any of the foregoing or related aspects, wherein the patient is chronically administered casimersen. In some aspects, the patient is administered casimersen for at least 48 weeks. In some aspects, the patient is administered casimersen for more than one year, more than two years, more than three years, more than four years, more than five years, more than ten years, more than twenty years, or more than thirty years.

In some aspects, the disclosure provides methods according to any of the foregoing or related aspects, wherein the patient is on a stable dose of corticosteroids for at least 6 months prior to administration of casimersen. In some aspects, the patient is on a stable dose of corticosteroids for at least 6 months prior to administration of casimersen and remains on corticosteroids during administration of casimersen.

In some aspects, the disclosure provides methods according to any of the foregoing or related aspects, wherein casimersen or a pharmaceutically acceptable salt thereof is formulated as a pharmaceutical composition. In some aspects, casimersen or a pharmaceutically acceptable salt thereof is formulated as a pharmaceutical composition having a strength of 50 mg/mL. In some aspects, casimersen or a pharmaceutically acceptable salt thereof is formulated as a pharmaceutical composition having a strength of 50 mg/mL and presented in a dosage form of 100 mg/2 mL. In some aspects, casimersen or a pharmaceutically acceptable salt thereof is formulated as a pharmaceutical composition having a strength of 50 mg/mL and presented in a dosage form of 500 mg/2 mL. In some aspects, the dosage form is contained in a single-use vial.

In some aspects, casimersen or a pharmaceutically acceptable salt thereof is formulated as a pharmaceutical composition comprising casimersen or a pharmaceutically acceptable salt thereof and a pharmaceutically acceptable carrier. In some aspects, the pharmaceutically acceptable carrier is a phosphate-buffered solution.

In some aspects, the disclosure provides a method according to any of the foregoing or related aspects, wherein exon skipping is measured by reverse transcription polymerase chain reaction (RT-PCR).

In some aspects, the disclosure provides a method according to any of the foregoing or related aspects, wherein the method increases dystrophin production in the patient. In some aspects, the dystrophin production is measured by western blot analysis. In some aspects, the dystrophin production is measured by immunohistochemistry (IHC).

In some aspects, the disclosure provides a method according to any of the foregoing or related aspects, further comprising confirming that the patient has a mutation in the DMD gene that is amenable to exon 45 skipping prior to administering casimersen.

In some aspects, the disclosure provides casimersen or a pharmaceutically acceptable salt thereof for use in treating Duchenne muscular dystrophy (DMD) in a patient in need thereof, the patient having a mutation of the DMD gene that is amenable to exon 45 skipping, wherein the treatment comprises administering to the patient a single intravenous dose of casimersen of about 30 mg/kg once weekly.

In some aspects, the disclosure provides casimersen or a pharmaceutically acceptable salt thereof for use in restoring an mRNA reading frame to induce exon skipping in a patient with Duchenne muscular dystrophy (DMD) in need thereof, the patient having a mutation of the DMD gene that is amenable to exon 45 skipping, wherein the treatment comprises administering to the patient a single intravenous dose of casimersen of about 30 mg/kg once weekly.

In some aspects, the disclosure provides casimersen or a pharmaceutically acceptable salt thereof for use in increasing dystrophin production in a patient with Duchenne muscular dystrophy (DMD) in need thereof, the patient having a mutation of the DMD gene that is amenable to exon 45 skipping, wherein the treatment comprises administering to the patient a single intravenous dose of casimersen of about 30 mg/kg once weekly.

### DETAILED DESCRIPTION

Embodiments of the present disclosure relate to methods for treating muscular dystrophy, such as DMD, by administering an antisense oligonucleotide specifically designed to induce exon 45 skipping in the human dystrophin gene, casimersen. Dystrophin plays a vital role in muscle function, and various muscle-related diseases are characterized by mutated forms of this gene. Hence, in certain embodiments, the methods described herein may be used for inducing exon 45 skipping in mutated forms of the human dystrophin gene, such as the mutated dystrophin genes found in DMD.

Thus, the disclosure relates to methods for treating muscular dystrophy, such as DMD, by inducing exon 45 skipping in a patient. Furthermore, the disclosure relates to methods for restoring an mRNA reading frame to induce exon skipping in a patient with DMD. The disclosure also relates to methods for increasing dystrophin production in a patient with DMD.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

### I. Definitions

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

“Amenable to exon 45 skipping” as used herein with regard to a subject or patient is intended to include subjects and patients having one or more mutations in the dystrophin gene which, absent the skipping of exon 45 of the dystrophin gene, causes the reading frame to be out-of-frame thereby disrupting translation of the pre-mRNA leading to an inability of the subject or patient to produce dystrophin. Non-limiting examples of mutations in the following exons of the dystrophin gene are amenable to exon 45 skipping include, e.g., deletion of: exons 7 to 42, 12 to 42, 18 to 42, 44 to 46, 44 to 47, 44 to 48, 44 to 49, 44 to 51, 44 to 53, 44 to 55, 44 to 57, or 44 to 59, or exon 44. Determining whether a patient has a mutation in the dystrophin gene that is amenable to exon skipping is well within the purview of one of skill in the art (see, e.g., Aartsma-Rus et al. (2009) Hum Mutat. 30:293-299, Gurvich et al., Hum Mutat. 2009; 30(4) 633-640, and Fletcher et al. (2010) Molecular Therapy 18(6) 1218-1223).

The terms “antisense oligomer” and “antisense compound” and “antisense oligonucleotide” and “oligomer” and “oligonucleotide” are used interchangeably in this disclosure and refer to a sequence of cyclic subunits connected by intersubunit linkages, with each cyclic subunit consisting of: (i) a ribose sugar or a derivative thereof; and (ii) a base-pairing moiety bound thereto, such that the order of the base-pairing moieties forms a base sequence that is complementary to a target sequence in a nucleic acid (typically an RNA) by Watson-Crick base pairing, to form a nucleic acid: oligomer heteroduplex within the target sequence. In certain embodiments, the oligomer is a phosphorodiamidate morpholino oligomer (“PMO”). In other embodiments, the antisense oligonucleotide is a 2′-O-methyl phosphorothioate. In other embodiments, the antisense oligonucleotide of the disclosure is a peptide nucleic acid (PNA), a locked nucleic acid (LNA), or a bridged nucleic acid (BNA) such as 2′-O,4′-C-ethylene-bridged nucleic acid (ENA).

The terms “complementary” and “complementarity” refer to two or more oligomers (i.e., each comprising a nucleobase sequence) that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some embodiments, complementarity between a given nucleobase sequence and the other nucleobase sequence may be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

“Dystrophin” is a rod-shaped cytoplasmic protein, and a vital part of the protein complex that connects the cytoskeleton of a muscle fiber to the surrounding extracellular matrix through the cell membrane. Dystrophin contains multiple functional domains. For instance, dystrophin contains an actin binding domain at about amino acids 14-240 and a central rod domain at about amino acids 253-3040. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a Glycine or Proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360), including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685.

The amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. The DAPC is destabilized when dystrophin is absent, which results in diminished levels of the member proteins, and in turn leads to progressive fibre damage and membrane leakage. In various forms of muscular dystrophy, such as Duchenne muscular dystrophy (DMD) and Becker muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in certain subjects with DMD or BMD, as known in the art, or by the absence of detectable dystrophin.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human dystrophin gene has about 79 exons.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

An “effective amount” or “therapeutically effective amount” refers to an amount of therapeutic compound, such as an antisense oligomer including, for example, casimersen, administered to a mammalian subject, either as a single dose or as part of a series of doses, which is effective to produce a desired therapeutic effect. For an antisense oligomer, this effect is can be brought about by inhibiting translation or natural splice-processing of a selected target sequence, or exon skipping to increase production of dystrophin.

In some embodiments, an effective amount is at least 4 mg/kg, at least 10 mg/kg or at least 20 mg/kg of an antisense oligomer, or a composition including an antisense oligomer, for a period of time to treat the subject. In some embodiments, an effective amount is at least 4 mg/kg, at least 10 mg/kg or at least 20 mg/kg of an antisense oligomer, or a composition including an antisense oligomer, to increase the number of dystrophin-positive fibers in a subject. In various embodiments, an effective amount is at least 4 mg/kg, at least 10 mg/kg about 10 mg/kg to about 20 mg/kg, about 20 mg/kg to about 30 mg/kg, about 25 mg/kg to about 30 mg/kg, or about 30 mg/kg to about 50 mg/kg. In some embodiments, an effective amount is about 30 mg/kg or about 50 mg/kg.

In various embodiments, an effective amount is at least 4 mg/kg, at least 10 mg/kg or at least 20 mg/kg of an antisense oligomer, or a composition including an antisense oligomer, to increase the production of dystrophin in a subject. In various embodiments, an effective amount is at least 4 mg/kg, at least 10 mg/kg, about 10 mg/kg to about 20 mg/kg, about 20 mg/kg to about 30 mg/kg, about 25 mg/kg to about 30 mg/kg, or about 30 mg/kg to about 50 mg/kg. In some embodiments, an effective amount is about 30 mg/kg or about 50 mg/kg.

In certain embodiments, an effective amount is at least 4 mg/kg, at least 10 mg/kg or at least 20 mg/kg of an antisense oligomer, or a composition including an antisense oligomer, to stabilize, maintain, or improve walking distance from a 20% deficit, for example in a 6 MWT, in a patient, relative to a healthy peer. In various embodiments, an effective amount is at least 4 mg/kg, at least 10 mg/kg, about 10 mg/kg to about 20 mg/kg, about 20 mg/kg to about 30 mg/kg, about 25 mg/kg to about 30 mg/kg, or about 30 mg/kg to about 50 mg/kg. In some embodiments, an effective amount is about 30 mg/kg or about 50 mg/kg.

In certain embodiments, an effective amount is at least 4 mg/kg, at least 10 mg/kg, about 10 mg/kg, about 20 mg/kg, about 25 mg/kg, about 30 mg/kg, or about 30 mg/kg to about 50 mg/kg, for at least 24 weeks, at least 36 weeks, or at least 48 weeks, to thereby increase the number of dystrophin-positive fibers in a subject. In certain embodiments, the increase in dystrophin-positive fibers in the subject is to at least 20%, about 30%, about 40%, about 50%, about 60%, about 70%, about 80%, about 90%, about 95% of normal. In some embodiments, treatment increases the number of dystrophin-positive fibers to 20-60%, or 30-50% of normal in the patient.

In certain embodiments, an effective amount is at least 4 mg/kg, at least 10 mg/kg, about 10 mg/kg, about 20 mg/kg, about 25 mg/kg, about 30 mg/kg, or about 30 mg/kg to about 50 mg/kg, for at least 24 weeks, at least 36 weeks, or at least 48 weeks, to and stabilize or improve walking distance from a 20% deficit, for example in a 6 MWT, in the patient relative to a healthy peer.

In various embodiments, an effective amount is at least 4 mg/kg, at least 10 mg/kg, about 10 mg/kg, about 20 mg/kg, about 25 mg/kg, about 30 mg/kg, or about 30 mg/kg to about 50 mg/kg, for at least 24 weeks, at least 36 weeks, or at least 48 weeks, to thereby increase dystrophin production in the patient. In some embodiments, the increased dystrophin production is about 0.1%, 0.2% 0.3% 0.5%, 0.7%, 0.9%, 1%, 1.01%, 1.5%, 2%, 2.01%, 2.5%, 3%, 3.01%, 3.5%, 4%, 4.01%, 4.5%, 5%, 5.01%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 27%, 28%, 29%, 30%, 31%, 32%, 33%, 34%, 35%, 40%, 45%, 50%, 55%, or 60% relative to a healthy peer. In certain embodiments, the increase in dystrophin production can be by about 0.1% to 0.5%, 0.5% to 0.9%, 0.8% to 1%, 0.9% to 1.2%, 0.9% to 1.0%, 0.9% to 1.01%, 1% to 1.01%, 1% to 1.5%, 1.5% to 2%, 1.9% to 2.0%, 1.9% to 2.01%, 2% to 2.01%, 2%, to 2.5%, 2.5% to 3%, 2.9% to 3.0%, 2.9% to 3.01%, 2% to 3.01%, 3% to 3.5%, 3.5% to 4%, 4% to 4.5%, 4.5% to 5%, 5% to 6%, 6% to 7%, 7% to 8%, 8% to 9%, 9% to 10%, 1% to 2%, 1% to 3%, 1% to 5%, 2% to 4%, 2% to 5%, 4% to 6%, 5%, to 8%, 8% to 10%, 1% to 5%, 2% to 6%, 3% to 7%, 4% to 8%, 5% to 10%, 10% to 12%, 12% to 15%, 15% to 20%, 17% to 20%, 20% to 22%, 20% to 25%, 25% to 30%, or 30% to 35% relative to a healthy peer.

By “enhance” or “enhancing,” or “increase” or “increasing,” or “stimulate” or “stimulating,” refers generally to the ability of one or more antisense oligonucleotides including, for example, casimersen, or pharmaceutical compositions thereof to produce or cause a greater physiological response (i.e., downstream effects) in a cell or a subject, as compared to the response caused by either no antisense oligonucleotide or a control compound. A measurable physiological response may include increased expression of (or production of) a functional form of a dystrophin protein, or increased dystrophin-related biological activity in muscle tissue, among other responses apparent from the understanding in the art and the description herein. Increased muscle function can also be measured, including increases or improvements in muscle function by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. The percentage of muscle fibres that express a functional dystrophin can also be measured, including increased dystrophin expression in about 1%, 2%, 5%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of muscle fibres. For instance, it has been shown that around 40% of muscle function improvement can occur if 25-30% of fibers express dystrophin (see, e.g., DelloRusso et al, Proc Natl Acad Sci USA 99: 12979-12984, 2002). In some embodiments, the increased dystrophin production is about 0.1%, 0.2% 0.3% 0.5%, 0.7%, 0.9%, 1%, 1.01%, 1.5%, 2%, 2.01%, 2.5%, 3%, 3.01%, 3.5%, 4%, 4.01%, 4.5%, 5%, 5.01%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 27%, 28%, 29%, 30%, 31%, 32%, 33%, 34%, 35%, 40%, 45%, 50%, 55%, or 60% relative to a healthy peer. In certain embodiments, the increase in dystrophin production can be by about 0.1% to 0.5%, 0.5% to 0.9%, 0.8% to 1%, 0.9% to 1.2%, 0.9% to 1.0%, 0.9% to 1.01%, 1% to 1.01%, 1% to 1.5%, 1.5% to 2%, 1.9% to 2.0%, 1.9% to 2.01%, 2% to 2.01%, 2%, to 2.5%, 2.5% to 3%, 2.9% to 3.0%, 2.9% to 3.01%, 2% to 3.01%, 3% to 3.5%, 3.5% to 4%, 4% to 4.5%, 4.5% to 5%, 5% to 6%, 6% to 7%, 7% to 8%, 8% to 9%, 9% to 10%, 1% to 2%, 1% to 3%, 1% to 5%, 2% to 4%, 2% to 5%, 4% to 6%, 5%, to 8%, 8% to 10%, 1% to 5%, 2% to 6%, 3% to 7%, 4% to 8%, 5% to 10%, 10% to 12%, 12% to 15%, 15% to 20%, 17% to 20%, 20% to 22%, 20% to 25%, 25% to 30%, or 30% to 35% relative to a healthy peer. As used herein, “increased dystrophin production,” “an increase in the production of dystrophin,” or the like refers to an increase in production of at least one of dystrophin, a dystrophin-like protein, or a functional dystrophin protein in the subject.

An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1), e.g., 1.5, 1.6, 1.7, 1.8, etc.) the amount produced by no antisense oligonucleotide (the absence of an agent) or a control compound.

The term “reduce” or “inhibit” may relate generally to the ability of one or more antisense compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of muscular dystrophy, or reductions in the expression of defective forms of dystrophin, such as the altered forms of dystrophin that are expressed in individuals with DMD or BMD. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain subjects with DMD or BMD. In certain embodiments, a functional dystrophin protein may have about 10%, 20%, 30%, 40%, 50%, 60%, 70%, 80%, 90%, or 100% (including all integers in between) of the in vitro or in vivo biological activity of wild-type dystrophin, as measured according to routine techniques in the art. As one example, dystrophin-related activity in muscle cultures in vitro can be measured according to myotube size, myofibril organization (or disorganization), contractile activity, and spontaneous clustering of acetylcholine receptors (see, e.g., Brown et al., Journal of Cell Science. 112:209-216, 1999). Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative (see, e.g., Collins & Morgan, Int J Exp Pathol 84: 165-172, 2003). These and other animal models can be used to measure the functional activity of various dystrophin proteins. Included are truncated forms of dystrophin, such as those forms that are produced by certain of the exon-skipping antisense oligonucleotides of the present disclosure.

The terms “morpholino,” “morpholino oligomer,” or “PMO” refer to a phosphorodiamidate morpholino oligomer of the following general structure:

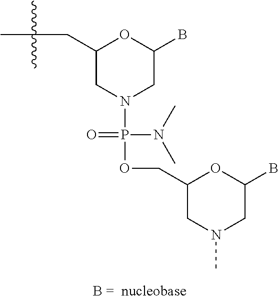

and as described in FIG. 2 of Summerton, J., et al., Antisense & Nucleic Acid Drug Development, 7: 187-195 (1997). Morpholinos as described herein are intended to cover all stereoisomers (and mixtures thereof) and configurations of the foregoing general structure. The synthesis, structures, and binding characteristics of morpholino oligomers are detailed in U.S. Pat. Nos. 5,698,685, 5,217,866, 5,142,047, 5,034,506, 5,166,315, 5,521,063, 5,506,337, 8,076,476, and 8,299,206, all of which are incorporated herein by reference. In certain embodiments, a morpholino is conjugated at the 5′ or 3′ end of the oligomer with a “tail” moiety to increase its stability and/or solubility. Exemplary tails include:
(1)

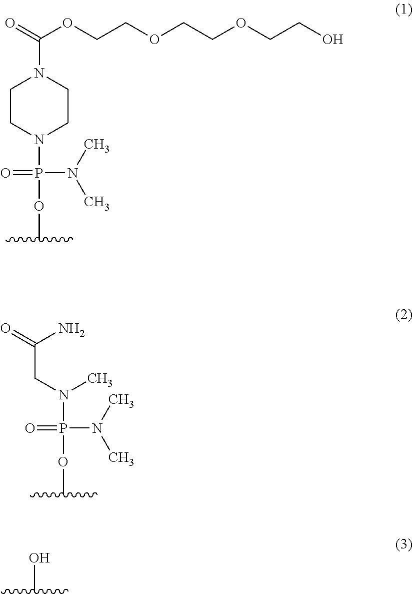

“Casimersen”, also known by its code name “SRP-4045” is a PMO having the base sequence 5′-CAATGCCATCCTGGAGTTCCTG-3′ (SEQ ID NO:1). Casimersen is registered under CAS Registry Number 1422959-91-8. Chemical names include: all-P-ambo-[P,2′,3′-trideoxy-P-(dimethylamino)-2′,3′-imino-2′,3′-seco](2′a→5′)(C-A-A-T-G-C-C-A-T-C-C-T-G-G-A-G-T-T-C-C-T-G) 5′-[4-({2-[2-(2-hydroxyethoxy)ethoxy]ethoxy}carbonyl)-N,N-dimethylpiperazine-1-phosphonamidate] (SEQ ID NO:1).

Casimersen has the following structure:

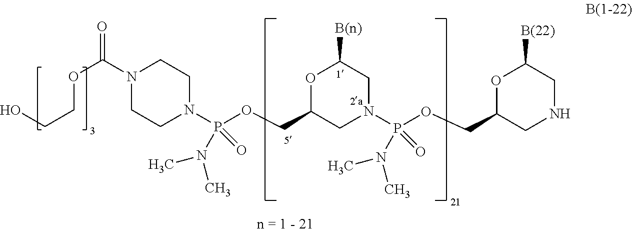

(SEQ ID NO: 1)
C-A-A-T-G-C-C-A-T-C-C-T-G-G-A-G-T-T-C-C-T-G

And also is represented by the following chemical structure:

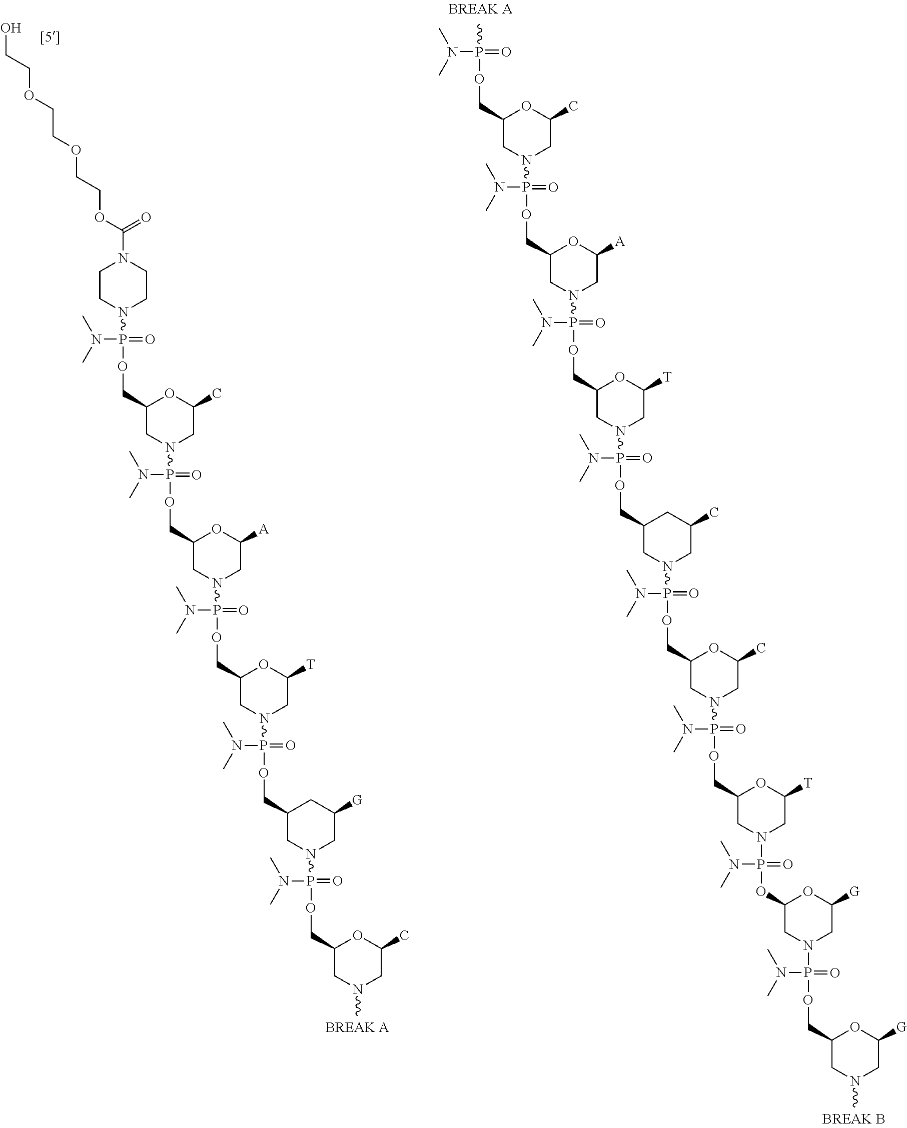

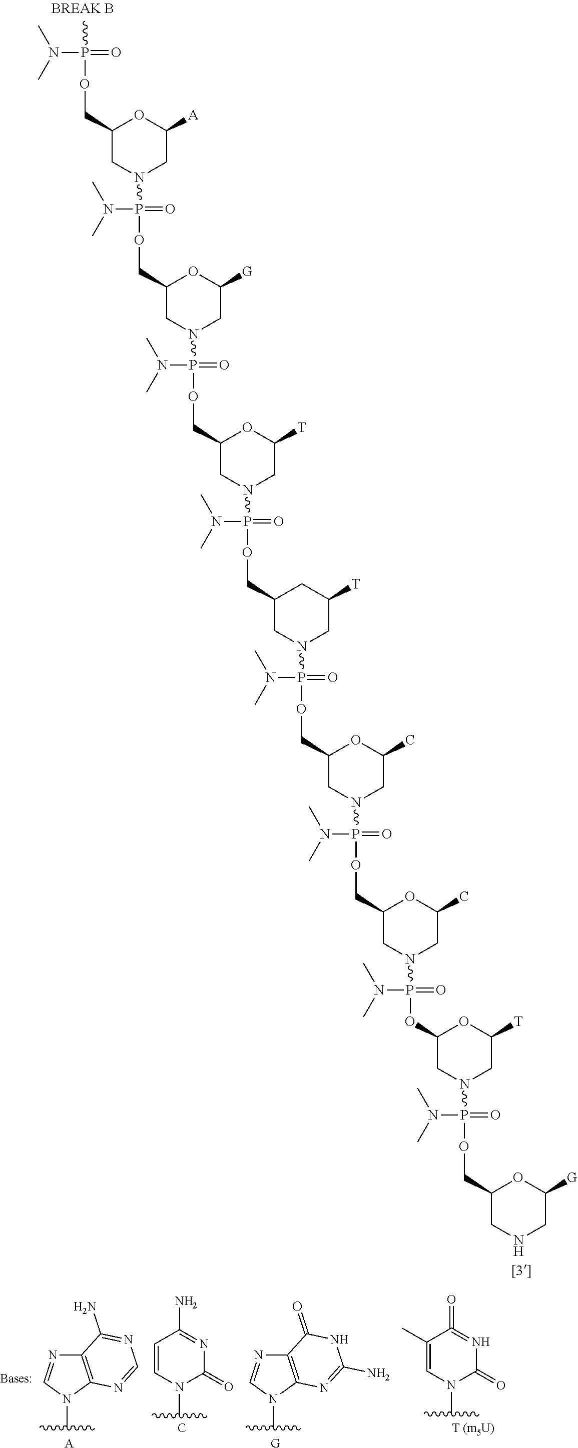

For clarity, structures of the disclosure including, for example, the above structure of casimersen, are continuous from 5′ to 3′, and, for the convenience of depicting the entire structure in a compact form, various illustration breaks labeled “BREAK A” and “BREAK B” have been included. As would be understood by the skilled artisan, for example, each indication of “BREAK A” shows a continuation of the illustration of the structure at these points. The skilled artisan understands that the same is true for each instance of “BREAK B” in the structures above. None of the illustration breaks, however, are intended to indicate, nor would the skilled artisan understand them to mean, an actual discontinuation of the structure above.

As used herein, a set of brackets used within a structural formula indicate that the structural feature between the brackets is repeated. In some embodiments, the brackets used can be “[” and “],” and in certain embodiments, brackets used to indicate repeating structural features can be “(” and “).” In some embodiments, the number of repeat iterations of the structural feature between the brackets is the number indicated outside the brackets such as 2, 3, 4, 5, 6, 7, and so forth. In various embodiments, the number of repeat iterations of the structural feature between the brackets is indicated by a variable indicated outside the brackets such as “Z”.

As used herein, a bond draw to chiral carbon or phosphorous atom within a straight bond or a squiggly bond structural formula indicates that the stereochemistry of the chiral carbon or phosphorous is undefined and is intended to include all forms of the chiral center. Examples of such illustrations are depicted below:

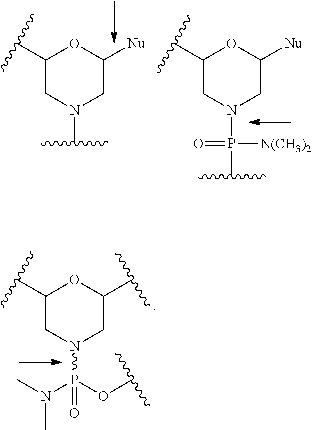

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” means the substance or composition must be compatible, chemically and/or toxicologically, with the other ingredients comprising a formulation, and/or the subject being treated therewith.

The phrase “pharmaceutically-acceptable carrier” as used herein means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The term “restoration” of dystrophin synthesis or production refers generally to the production of a dystrophin protein including truncated forms of dystrophin in a patient with muscular dystrophy following treatment with an antisense oligomer as described herein. In some embodiments, treatment results in an increase in dystrophin production in a patient by 1%, 5%, 10%, 20%, 30%, 40%, 50%, 60%, 70%, 80%, 90% or 100% (including all integers in between). In some embodiments, treatment increases the number of dystrophin-positive fibers to at least 20%, about 30%, about 40%, about 50%, about 60%, about 70%, about 80%, about 90% or about 95% to 100% of normal in the subject. In other embodiments, treatment increases the number of dystrophin-positive fibers to about 20% to about 60%, or about 30% to about 50% of normal in the subject. The percent of dystrophin-positive fibers in a patient following treatment can be determined by a muscle biopsy using known techniques. For example, a muscle biopsy may be taken from a suitable muscle, such as the biceps brachii muscle in a patient.

Analysis of the percentage of positive dystrophin fibers may be performed pre-treatment and/or post-treatment or at time points throughout the course of treatment. In some embodiments, a post-treatment biopsy is taken from the contralateral muscle from the pre-treatment biopsy. Pre- and post-treatment dystrophin expression studies may be performed using any suitable assay for dystrophin. In some embodiments, immunohistochemical detection is performed on tissue sections from the muscle biopsy using an antibody that is a marker for dystrophin, such as a monoclonal or a polyclonal antibody. For example, the MANDYS106 antibody can be used which is a highly sensitive marker for dystrophin. Any suitable secondary antibody may be used.

In some embodiments, the percent dystrophin-positive fibers are calculated by dividing the number of positive fibers by the total fibers counted. Normal muscle samples have 100% dystrophin-positive fibers. Therefore, the percent dystrophin-positive fibers can be expressed as a percentage of normal. To control for the presence of trace levels of dystrophin in the pretreatment muscle as well as revertant fibers a baseline can be set using sections of pre-treatment muscles from each patient when counting dystrophin-positive fibers in post-treatment muscles. This may be used as a threshold for counting dystrophin-positive fibers in sections of post-treatment muscle in that patient. In other embodiments, antibody-stained tissue sections can also be used for dystrophin quantification using Bioquant image analysis software (Bioquant Image Analysis Corporation, Nashville, Tenn.). The total dystrophin fluorescence signal intensity can be reported as a percentage of normal. In addition, Western blot analysis with monoclonal or polyclonal anti-dystrophin antibodies can be used to determine the percentage of dystrophin positive fibers. For example, the anti-dystrophin antibody NCL-Dys1 from Novacastra may be used. The percentage of dystrophin-positive fibers can also be analyzed by determining the expression of the components of the sarcoglycan complex (β,γ) and/or neuronal NOS.

In some embodiments, treatment with an antisense oligomer of the disclosure, such as casimersen, slows or reduces the progressive respiratory muscle dysfunction and/or failure in patients with DMD that would be expected without treatment. In some embodiments, treatment with an antisense oligomer of the disclosure may reduce or eliminate the need for ventilation assistance that would be expected without treatment. In some embodiments, measurements of respiratory function for tracking the course of the disease, as well as the evaluation of potential therapeutic interventions include Maximum inspiratory pressure (MIP), maximum expiratory pressure (MEP) and forced vital capacity (FVC). MIP and MEP measure the level of pressure a person can generate during inhalation and exhalation, respectively, and are sensitive measures of respiratory muscle strength. MIP is a measure of diaphragm muscle weakness.

In some embodiments, MEP may decline before changes in other pulmonary function tests, including MIP and FVC. In certain embodiments, MEP may be an early indicator of respiratory dysfunction. In certain embodiments, FVC may be used to measure the total volume of air expelled during forced exhalation after maximum inspiration. In patients with DMD, FVC increases concomitantly with physical growth until the early teens. However, as growth slows or is stunted by disease progression, and muscle weakness progresses, the vital capacity enters a descending phase and declines at an average rate of about 8 to 8.5 percent per year after 10 to 12 years of age. In certain embodiments, MIP percent predicted (MIP adjusted for weight), MEP percent predicted (MEP adjusted for age) and FVC percent predicted (FVC adjusted for age and height) are supportive analyses.

A “subject” or “patient,” as used herein, includes any animal that exhibits a symptom, or is at risk for exhibiting a symptom, which can be treated with an antisense oligonucleotide of the disclosure, such as a subject that has or is at risk for having DMD or BMD, or any of the symptoms associated with these conditions (e.g., muscle fibre loss). Suitable subjects (patients) include laboratory animals (such as mouse, rat, rabbit, or guinea pig), farm animals, and domestic animals or pets (such as a cat or dog). Non-human primates and, preferably, human patients, are included. Also included are methods of producing dystrophin in a subject having a mutation of the dystrophin gene that is amenable to exon 45 skipping.

A “pediatric patient” as used herein is a patient from age 1 to 21, inclusive.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

“Chronic administration,” as used herein, refers to continuous, regular, long-term therapeutic administration, i.e., periodic administration without substantial interruption. For example, daily, for a period of time of at least several weeks or months or years, for the purpose of treating muscular dystrophy in a patient. For example, weekly, for a period of time of at least several months or years, for the purpose of treating muscular dystrophy in a patient (e.g., weekly for at least six weeks, weekly for at least 12 weeks, weekly for at least 24 weeks, weekly for at least 48 weeks, weekly for at least 72 weeks, weekly for at least 96 weeks, weekly for at least 120 weeks, weekly for at least 144 weeks, weekly for at least 168 weeks, weekly for at least 180 weeks, weekly for at least 192 weeks, weekly for at least 216 weeks, or weekly for at least 240 weeks).

“Periodic administration,” as used herein, refers to administration with an interval between doses. For example, periodic administration includes administration at fixed intervals (e.g., weekly, monthly) that may be recurring.

“Placebo,” as used herein, refers to a substance that has no therapeutic effect and may be used as a control.

“Placebo control,” as used herein, refers to a subject or patient that receives a placebo rather than the combination therapy, antisense oligonucleotide, non-steroidal anti-inflammatory compound, and/or another pharmaceutical composition. The placebo control may have the same mutation status, be of similar age, similar ability to ambulate, and or receive the same concomitant medications (including steroids, etc.), as the subject or patient.

The phase “targeting sequence,” “base sequence,” or “nucleobase sequence” refers to a sequence of nucleobases of an oligomer that is complementary to a sequence of nucleotides in a target pre-mRNA. In some embodiments of the disclosure, the sequence of nucleotides in the target pre-mRNA is an exon 45 annealing site in the dystrophin pre-mRNA designated as H45A(−03+19).

“Treatment” of a subject (e.g. a mammal, such as a human) or a cell is any type of intervention used in an attempt to alter the natural course of the subject or cell. Treatment includes, but is not limited to, administration of an oligomer or a pharmaceutical composition thereof, and may be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

In some embodiments, treatment with an antisense oligomer of the disclosure increases dystrophin production, delays disease progression, slows or reduces the loss of ambulation, reduces muscle inflammation, reduces muscle damage, improves muscle function, reduces loss of pulmonary function, and/or enhances muscle regeneration that would be expected without treatment or that would be expected without treatment. In some embodiments, treatment maintains, delays, or slows disease progression. In some embodiments, treatment maintains ambulation or reduces the loss of ambulation. In some embodiments, treatment maintains pulmonary function or reduces loss of pulmonary function. In some embodiments, treatment maintains or increases a stable walking distance in a patient, as measured by, for example, the 6 Minute Walk Test (6MWT). In some embodiments, treatment maintains or reduces the time to walk/run 10 meters (i.e., the 10 meter walk/run test). In some embodiments, treatment maintains or reduces the time to stand from supine (Le, time to stand test). In some embodiments, treatment maintains or reduces the time to climb four standard stairs (i.e., the four-stair climb test). In some embodiments, treatment maintains or reduces muscle inflammation in the patient, as measured by, for example, MRI (e.g., MRI of the leg muscles). In some embodiments, MRI measures T2 and/or fat fraction to identify muscle degeneration. MRI can identify changes in muscle structure and composition caused by inflammation, edema, muscle damage and fat infiltration.

In some embodiments, treatment with an antisense oligomer of the disclosure increases dystrophin production. In some embodiments, the increased dystrophin production is about 0.1%, 0.2% 0.3% 0.5%, 0.7%, 0.9%, 1%, 1.01%, 1.5%, 2%, 2.01%, 2.5%, 3%, 3.01%, 3.5%, 4%, 4.01%, 4.5%, 5%, 5.01%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 27%, 28%, 29%, 30%, 31%, 32%, 33%, 34%, 35%, 40%, 45%, 50%, 55%, or 60% relative to a healthy peer. In certain embodiments, the increase in dystrophin production can be by about 0.1% to 0.5%, 0.5% to 0.9%, 0.8% to 1%, 0.9% to 1.2%, 0.9% to 1.0%, 0.9% to 1.01%, 1% to 1.01%, 1% to 1.5%, 1.5% to 2%, 1.9% to 2.0%, 1.9% to 2.01%, 2% to 2.01%, 2%, to 2.5%, 2.5% to 3%, 2.9% to 3.0%, 2.9% to 3.01%, 2% to 3.01%, 3% to 3.5%, 3.5% to 4%, 4% to 4.5%, 4.5% to 5%, 5% to 6%, 6% to 7%, 7% to 8%, 8% to 9%, 9% to 10%, 1% to 2%, 1% to 3%, 1% to 5%, 2% to 4%, 2% to 5%, 4% to 6%, 5%, to 8%, 8% to 10%, 1% to 5%, 2% to 6%, 3% to 7%, 4% to 8%, 5% to 10%, 10% to 12%, 12% to 15%, 15% to 20%, 17% to 20%, 20% to 22%, 20% to 25%, 25% to 30%, or 30% to 35% relative to a healthy peer.

In certain embodiments, treatment with an antisense oligomer of the disclosure increases dystrophin production and slows or reduces the loss of ambulation that would be expected without treatment. For example, treatment may stabilize, maintain, improve or increase walking ability (e.g., stabilization of ambulation) in the subject. In some embodiments, treatment maintains or increases a stable walking distance in a patient, as measured by, for example, the 6 Minute Walk Test (6MWT), described by McDonald, et al. (Muscle Nerve, 2010; 42:966-74, herein incorporated by reference). A change in the 6 Minute Walk Distance (6MWD) may be expressed as an absolute value, a percentage change or a change in the %-predicted value. In some embodiments, treatment maintains or improves a stable walking distance in a 6MWT from a 20% deficit in the subject relative to a healthy peer. The performance of a DMD patient in the 6MWT relative to the typical performance of a healthy peer can be determined by calculating a %-predicted value. For example, the %-predicted 6MWD may be calculated using the following equation for males: 196.72+(39.81×age)−(1.36×age²)+(132.28×height in meters). For females, the %-predicted 6MWD may be calculated using the following equation: 188.61+(51.50×age)−(1.86×age²)+(86.10×height in meters) (Henricson et al. PLoS Curr., 2012, version 2, herein incorporated by reference).

In some embodiments, treatment with an antisense oligomer increases the stable walking distance in the patient from baseline to greater than 3, 5, 6, 7, 8, 9, 10, 15, 20, 25, 30 or 50 meters (including all integers in between). In some embodiments, the increased dystrophin production is about 0.1%, 0.2% 0.3% 0.5%, 0.7%, 0.9%, 1%, 1.01%, 1.5%, 2%, 2.01%, 2.5%, 3%, 3.01%, 3.5%, 4%, 4.01%, 4.5%, 5%, 5.01%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 27%, 28%, 29%, 30%, 31%, 32%, 33%, 34%, 35%, 40%, 45%, 50%, 55%, or 60% relative to a healthy peer. In certain embodiments, the increase in dystrophin production can be by about 0.1% to 0.5%, 0.5% to 0.9%, 0.8% to 1%, 0.9% to 1.2%, 0.9% to 1.0%, 0.9% to 1.01%, 1% to 1.01%, 1% to 1.5%, 1.5% to 2%, 1.9% to 2.0%, 1.9% to 2.01%, 2% to 2.01%, 2%, to 2.5%, 2.5% to 3%, 2.9% to 3.0%, 2.9% to 3.01%, 2% to 3.01%, 3% to 3.5%, 3.5% to 4%, 4% to 4.5%, 4.5% to 5%, 5% to 6%, 6% to 7%, 7% to 8%, 8% to 9%, 9% to 10%, 1% to 2%, 1% to 3%, 1% to 5%, 2% to 4%, 2% to 5%, 4% to 6%, 5%, to 8%, 8% to 10%, 1% to 5%, 2% to 6%, 3% to 7%, 4% to 8%, 5% to 10%, 10% to 12%, 12% to 15%, 15% to 20%, 17% to 20%, 20% to 22%, 20% to 25%, 25% to 30%, or 30% to 35% relative to a healthy peer.

Loss of muscle function in patients with DMD may occur against the background of normal childhood growth and development. Indeed, younger children with DMD may show an increase in distance walked during 6MWT over the course of about 1 year despite progressive muscular impairment. In some embodiments, the 6MWD from patients with DMD is compared to typically developing control subjects and to existing normative data from age and sex matched subjects. In some embodiments, normal growth and development can be accounted for using an age and height based equation fitted to normative data. Such an equation can be used to convert 6MWD to a percent-predicted (%-predicted) value in subjects with DMD. In certain embodiments, analysis of %-predicted 6MWD data represents a method to account for normal growth and development, and may show that gains in function at early ages (e.g., less than or equal to age 7) represent stable rather than improving abilities in patients with DMD (Henricson et al. PLoS Curr., 2012, version 2, herein incorporated by reference).

An antisense molecule nomenclature system was proposed and published to distinguish between the different antisense molecules (see Mann et al., (2002) J Gen Med 4, 644-654). This nomenclature became especially relevant when testing several slightly different antisense molecules, all directed at the same target region, as shown below:

H #A/D(x:y).

The first letter designates the species (e.g. H: human, M: murine, C: canine). “#” designates target dystrophin exon number. “A/D” indicates acceptor or donor splice site at the beginning and end of the exon, respectively. (x y) represents the annealing coordinates where “−” or “+” indicate intronic or exonic sequences respectively. For example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense molecule. Entirely exonic annealing coordinates that would be represented by A(+65+85), that is the site between the 65th and 85th nucleotide from the start of that exon.

### II. Antisense Oligonucleotides

Antisense oligonucleotides that target the pre-mRNA of the dystrophin gene to effect the skipping of exon 45 are used accordance with the methods of this disclosure.

Such an antisense oligonucleotide can be designed to block or inhibit translation of mRNA or to inhibit natural pre-mRNA splice processing, and may be said to be “directed to” or “targeted against” a target sequence with which it hybridizes. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a preprocessed mRNA. In some embodiments, a target sequence may be any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

In certain embodiments, the antisense oligonucleotide specifically hybridizes to an exon 45 target region of the Dystrophin pre-mRNA and induces exon 45 skipping. In certain embodiments, the antisense oligonucleotide that hybridizes to an exon 45 target region of the Dystrophin pre-mRNA and induces exon 45 skipping is a Phosphorodiamidate Morpholino Oligomers (PMO).

In certain embodiments, the antisense oligonucleotide is casimersen.

Casimersen belongs to a distinct class of novel synthetic antisense RNA therapeutics called Phosphorodiamidate Morpholino Oligomers (PMO), which are a redesign of the natural nucleic acid structure. Casimersen is a PMO that hybridizes to an exon 45 target region of the Dystrophin pre-mRNA and induces exon 45 skipping. Casimersen can be prepared by stepwise solid-phase synthesis, employing methods detailed in the references cited above, and additionally in International Patent Application Serial No. PCT/US2017/040017 the entirety of which is expressly incorporated by reference herein.

PMOs offer potential clinical advantages based on in vivo nonclinical observations. PMOs incorporate modifications to the sugar ring of RNA that protect it from enzymatic degradation by nucleases in order to ensure stability in vivo. PMOs are distinguished from natural nucleic acids and other antisense oligonucleotide classes in part through the use of 6-membered synthetic morpholino rings, which replace the 5-membered ribofuranosyl rings found in RNA, DNA and many other synthetic antisense RNA oligonucleotides.

The uncharged phosphorodiamidate linkages specific to PMOs are considered to potentially confer reduced off-target binding to proteins. PMOs have an uncharged phosphorodiamidate linkage that links each morpholino ring instead of the negatively charged phosphorothioate linkage used in other clinical-stage synthetic antisense RNA oligonucleotides.

A potential therapeutic approach to the treatment of DMD caused by out-of-frame mutations in the DMD gene is suggested by the milder form of dystrophinopathy known as BMD, which is caused by in-frame mutations. The ability to convert an out-of-frame mutation to an in-frame mutation would hypothetically preserve the mRNA reading frame and produce an internally shortened yet functional dystrophin protein. Casimersen was designed to accomplish this.

Casimersen targets dystrophin pre-mRNA and induces skipping of exon 45, so it is excluded or skipped from the mature, spliced mRNA transcript. By skipping exon 45, the disrupted reading frame is restored to an in-frame mutation. While DMD is comprised of various genetic subtypes, casimersen was specifically designed to skip exon 45 of dystrophin pre-mRNA.

The sequence of casimersen's 22 nucleobases is designed to be complementary to a specific annealing site of dystrophin pre-mRNA and induce skipping of exon 45 during processing. Each morpholino ring in casimersen is linked to one of four heterocyclic nucleobases found in DNA (adenine, cytosine, guanine, and thymine).

Hybridization of casimersen with the targeted pre-mRNA sequence interferes with formation of the pre-mRNA splicing complex and deletes exon 45 from the mature mRNA. The structure and conformation of casimersen allows for sequence-specific base pairing to the complementary sequence. For example, eteplirsen, which is a PMO that was designed to skip exon 51 of dystrophin pre-mRNA allows for sequence-specific base pairing to the complementary sequence contained in exon 51 of dystrophin pre-mRNA.

Restoration of the Dystrophin Reading Frame Using Exon Skipping

Normal dystrophin mRNA containing all 79 exons will produce normal dystrophin protein.

Dystrophin mRNA missing whole exons from the dystrophin gene typically result in DMD.

Another exon skipping PMO, eteplirsen, skips exon 51 to restore the mRNA reading frame. Since exon 49 ends in a complete codon and exon 52 begins with the first nucleotide of a codon, deletion of exon 51 by exon skipping restores the reading frame, resulting in production of an internally-shortened dystrophin protein with an intact dystroglycan binding site.

The feasibility of ameliorating the DMD phenotype using exon skipping to restore the dystrophin mRNA open reading frame is supported by nonclinical research. Numerous studies in dystrophic animal models of DMD have shown that restoration of dystrophin by exon skipping leads to reliable improvements in muscle strength and function (Sharp 2011; Yokota 2009; Wu 2008; Wu 2011; Barton-Davis 1999; Goyenvalle 2004; Gregorevic 2006; Yue 2006; Welch 2007; Kawano 2008; Reay 2008; van Putten 2012). A compelling example of this comes from a study in which dystrophin levels following exon skipping (using a PMO) therapy were compared with muscle function in the same tissue. In dystrophic mdx mice, tibialis anterior (TA) muscles treated with a mouse-specific PMO maintained ˜75% of their maximum force capacity after stress-inducing contractions, whereas untreated contralateral TA muscles maintained only ˜25% of their maximum force capacity (p<0.05) (Sharp 2011). In another study, 3 dystrophic CXMD dogs received at (2-5 months of age) exon-skipping therapy using a PMO-specific for their genetic mutation once a week for 5 to 7 weeks or every other week for 22 weeks. Following exon-skipping therapy, all 3 dogs demonstrated extensive, body-wide expression of dystrophin in skeletal muscle, as well as maintained or improved ambulation (15 m running test) relative to baseline. In contrast, untreated age-matched CXMD dogs showed a marked decrease in ambulation over the course of the study (Yokota 2009).

PMOs were shown to have more exon skipping activity at equimolar concentrations than phosphorothioates in both mdx mice and in the humanized DMD (hDMD) mouse model, which expresses the entire human DMD transcript (Heemskirk 2009).

Clinical outcomes for analyzing the effect of an antisense oligonucleotide that specifically hybridizes to an exon 45 target region of the Dystrophin pre-mRNA and induces exon 45 skipping include an increase from baseline of percent dystrophin positive fibers (PDPF), six-minute walk test (6MWT), loss of ambulation (LOA), North Star Ambulatory Assessment (NSAA), pulmonary function tests (PFT), ability to rise (from a supine position) without external support, de novo dystrophin production and other functional measures.

### III. Formulations and Modes of Administration

In certain embodiments, the present disclosure provides formulations or pharmaceutical compositions suitable for the therapeutic delivery of antisense oligonucleotides, as described herein. Hence, in certain embodiments, the present disclosure provides pharmaceutically acceptable compositions that comprise a therapeutically-effective amount of one or more of the antisense oligonucleotides described herein, formulated together with one or more pharmaceutically acceptable carriers (additives) and/or diluents. While it is possible for an antisense oligonucleotide of the present disclosure to be administered alone, it is preferable to administer the compound as a pharmaceutical formulation (composition).

Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT WO 94/02595. These and other protocols can be utilized for the delivery of virtually any nucleic acid molecule, including the antisense oligonucleotides of the present disclosure.

As detailed below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the antisense oligonucleotides of the instant disclosure include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The disclosure also features the use of the composition comprising surface-modified liposomes containing poly (ethylene glycol) lipids (PEG-modified, branched and unbranched or combinations thereof, or long-circulating liposomes or stealth liposomes). Antisense oligonucleotides of the disclosure can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the present disclosure includes antisense oligonucleotide pharmaceutical compositions prepared for delivery as described in U.S. Pat. Nos. 6,692,911, 7,163,695 and 7,070,807. In this regard, in one embodiment, the present disclosure provides an oligomer of the present disclosure in a composition comprising copolymers of lysine and histidine (HK) (as described in U.S. Pat. Nos. 7,163,695, 7,070,807, and 6,692,911) either alone or in combination with PEG (e.g., branched or unbranched PEG or a mixture of both), in combination with PEG and a targeting moiety or any of the foregoing in combination with a crosslinking agent. In certain embodiments, the present disclosure provides antisense oligonucleotides in pharmaceutical compositions comprising gluconic-acid-modified polyhistidine or gluconylated-polyhistidine/transferrin-polylysine. One skilled in the art will also recognize that amino acids with properties similar to His and Lys may be substituted within the composition.

Certain embodiments of antisense oligonucleotides described herein may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present disclosure. These salts can be prepared in situ in the administration vehicle or the dosage form manufacturing process, or by separately reacting a purified compound of the disclosure in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the subject antisense oligonucleotides include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain embodiments, the antisense oligonucleotides of the present disclosure may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present disclosure. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present disclosure comprises an excipient selected from cyclodextrins, celluloses, liposomes, micelle forming agents, e.g., bile acids, and polymeric carriers, e.g., polyesters and polyanhydrides; and an oligomer of the present disclosure. In certain embodiments, an aforementioned formulation renders orally bioavailable an oligomer of the present disclosure.

Methods of preparing these formulations or pharmaceutical compositions include the step of bringing into association an antisense oligonucleotide of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. An antisense oligonucleotide of the present disclosure may also be administered as a bolus, electuary or paste.

In solid dosage forms of the disclosure for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid pharmaceutical compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid pharmaceutical compositions which can be dissolved in sterile water, or some other sterile injectable medium immediately before use. These pharmaceutical compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral pharmaceutical compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an oligomer as provided herein include powders, sprays, ointments, pastes, creams, lotions, gels, solutions, patches and inhalants. The active antisense oligonucleotides may be mixed under sterile conditions with a pharmaceutically-acceptable carrier, and with any preservatives, buffers, or propellants which may be required. The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an oligomer of the present disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an oligomer of the present disclosure to the body. Such dosage forms can be made by dissolving or dispersing the oligomer in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more antisense oligonucleotides of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These pharmaceutical compositions may also contain adjuvants such as preservatives, wetting agents, emulsifying agents and dispersing agents. Prevention of the action of microorganisms upon the subject antisense oligonucleotides may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of the subject antisense oligonucleotides in biodegradable polymers such as polylactide-polyglycolide. Depending on the ratio of oligomer to polymer, and the nature of the particular polymer employed, the rate of oligomer release can be controlled. Examples of other biodegradable polymers include poly(orthoesters) and poly(anhydrides). Depot injectable formulations may also prepared by entrapping the drug in liposomes or microemulsions that are compatible with body tissues.

When the antisense oligonucleotides of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

As noted above, the formulations or preparations of the present disclosure may be given orally, parenterally, topically, or rectally. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

Regardless of the route of administration selected, the antisense oligonucleotides of the present disclosure, which may be used in a suitable hydrated form, and/or the pharmaceutical compositions of the present disclosure, may be formulated into pharmaceutically-acceptable dosage forms by conventional methods known to those of skill in the art. Actual dosage levels of the active ingredients in the pharmaceutical compositions of this disclosure may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular oligomer of the present disclosure employed, or the ester, salt or amide thereof, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved. In general, a suitable daily dose of a compound of the disclosure will be that amount of the compound which is the lowest dose effective to produce a therapeutic effect. Such an effective dose will generally depend upon the factors described above. Generally, oral, intravenous, intracerebroventricular and subcutaneous doses of the compounds of this disclosure for a patient, when used for the indicated effects, will range from about 0.0001 to about 100 mg per kilogram of body weight per day.

In some embodiments, the antisense oligonucleotides of the present disclosure are administered in doses generally from about 4 to 100 mg/kg, about 10 to 100 mg/kg, or about 20 to 100 mg/kg. In some embodiments, parenteral doses such as, for example, i.v. administration are from about 0.5 mg to 100 mg/kg. In some embodiments, the antisense oligonucleotides are administered at doses of about 4 mg/kg, 10 mg/kg, 11 mg/kg, 12 mg/kg, 14 mg/kg, 15 mg/kg, 17 mg/kg, 20 mg/kg, 21 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 65 mg/kg, 70 mg/kg, 75 mg/kg, 80 mg/kg, 85 mg/kg, 90 mg/kg, 95 mg/kg, or 100 mg/kg, including all integers in between. In some embodiments, the oligomer is administered at about 4 mg/kg. In some embodiments, the oligomer is administered at about 10 mg/kg. In some embodiments, the oligomer is administered at about 20 mg/kg. In some embodiments, the oligomer is administered at about 30 mg/kg. In some embodiments, the oligomer is administered at about 40 mg/kg. In some embodiments, the oligomer is administered at about 50 mg/kg.

If desired, the effective daily dose of the active compound may be administered as two, three, four, five, six or more sub-doses administered separately at appropriate intervals throughout the day, optionally, in unit dosage forms. In certain situations, dosing is one administration per day. In certain embodiments, dosing is one or more administration per every 2, 3, 4, 5, 6, 7, 8, 9, 10, 11, 12, 13, 14 days, or every 1 or 2 weeks, as needed, to maintain the desired expression of a functional dystrophin protein. In certain embodiments, dosing is one administration once every week. In certain embodiments, dosing is one or more administrations once every two weeks. In some embodiments, dosing is one administration once every two weeks.

In various embodiments, the antisense oligonucleotides are administered weekly at about 4 mg/kg. In various embodiments, the antisense oligonucleotides are administered weekly at about 10 mg/kg. In various embodiments, the antisense oligonucleotides are administered weekly at about 20 mg/kg. In various embodiments, the antisense oligonucleotides are administered weekly at about 30 mg/kg. In some embodiments, the antisense oligonucleotides are administered weekly at about 40 mg/kg. In some embodiments, the antisense oligonucleotides are administered weekly at about 50 mg/kg. In some embodiments, the antisense oligonucleotides are administered weekly at about 60 mg/kg. In some embodiments, the antisense oligonucleotides are administered weekly at 80 mg/kg. As used herein, weekly is understood to have the art-accepted meaning of every week.

In various embodiments, the antisense oligonucleotides are administered biweekly at about 4 mg/kg. In various embodiments, the antisense oligonucleotides are administered biweekly at about 10 mg/kg. In various embodiments, the antisense oligonucleotides are administered biweekly at about 20 mg/kg. In various embodiments, the antisense oligonucleotides are administered biweekly at about 30 mg/kg. In some embodiments, the antisense oligonucleotides are administered biweekly at about 40 mg/kg. In some embodiments, the antisense oligonucleotides are administered biweekly at about 50 mg/kg. In some embodiments, the antisense oligonucleotides are administered biweekly at about 60 mg/kg. In some embodiments, the antisense oligonucleotides are administered biweekly at about 80 mg/kg. As used herein, biweekly is understood to have the art-accepted meaning of every two weeks.

Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

In one aspect of disclosure, the formulations contain micelles formed from an oligomer as provided herein and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present disclosure and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

In certain embodiments, the delivery may occur by use of liposomes, nanocapsules, microparticles, microspheres, lipid particles, vesicles, and the like, for the introduction of the pharmaceutical compositions of the present disclosure into suitable host cells. In particular, the pharmaceutical compositions of the present disclosure may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use in the present disclosure are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In certain embodiments, polymers have a molecular weight of from about 100 or 120 daltons up to about 5,000 or 10,000 daltons, or from about 300 daltons to about 5,000 daltons. In other embodiments, the polymer is polyethyleneglycol having a molecular weight of from about 100 to about 5,000 daltons, or having a molecular weight of from about 300 to about 5,000 daltons. In certain embodiments, the polymer is polyethyleneglycol of 750 daltons (PEG(750)). Polymers may also be defined by the number of monomers therein; a preferred embodiment of the present disclosure utilizes polymers of at least about three monomers, such PEG polymers consisting of three monomers (approximately 150 daltons).

Other hydrophilic polymers which may be suitable for use in the present disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letter α, β, or γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present disclosure relates to formulations comprising liposomes containing an antisense oligonucleotide of the present disclosure, where the liposome membrane is formulated to provide a liposome with increased carrying capacity. Alternatively or in addition, the compound of the present disclosure may be contained within, or adsorbed onto, the liposome bilayer of the liposome. An antisense oligonucleotide of the present disclosure may be aggregated with a lipid surfactant and carried within the liposome's internal space; in these cases, the liposome membrane is formulated to resist the disruptive effects of the active agent-surfactant aggregate.

According to one embodiment of the present disclosure, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present disclosure are in solubilized form. Aggregates of surfactant and active agent (such as emulsions or micelles containing the active agent of interest) may be entrapped within the interior space of liposomes according to the present disclosure. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the critical micelle concentration (“CMC”) of the surfactant and aids in micelle formation. Preferred are surfactants with CMCs in the micromolar range; higher CMC surfactants may be utilized to prepare micelles entrapped within liposomes of the present disclosure.

Liposomes according to the present disclosure may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993. For example, liposomes of the present disclosure may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present disclosure, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT® and Lipofectamine® may be utilized to introduce polynucleotides or proteins into cells.

The release characteristics of a formulation of the present disclosure depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

An antisense oligonucleotide may be formulated to be contained within, or, adapted to release by a surgical or medical device or implant. In certain aspects, an implant may be coated or otherwise treated with an antisense oligonucleotide. For example, hydrogels, or other polymers, such as biocompatible and/or biodegradable polymers, may be used to coat an implant with the pharmaceutical compositions of the present disclosure (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In addition to the methods provided herein, the antisense oligonucleotides for use according to the disclosure may be formulated for administration in any convenient way for use in human or veterinary medicine, by analogy with other pharmaceuticals. The antisense antisense oligonucleotides and their corresponding formulations may be administered alone or in combination with other therapeutic strategies in the treatment of muscular dystrophy, such as myoblast transplantation, stem cell therapies, administration of aminoglycoside antibiotics, proteasome inhibitors, and up-regulation therapies (e.g., upregulation of utrophin, an autosomal paralogue of dystrophin).

In some embodiments, the additional therapeutic may be administered prior, concurrently, or subsequently to the administration of the antisense oligonucleotide of the present disclosure. For example, the antisense oligonucleotides may be administered in combination with a steroid and/or antibiotic. In certain embodiments, the antisense oligonucleotides are administered to a patient that is on background steroid theory (e.g., intermittent or chronic/continuous background steroid therapy. For example, in some embodiments the patient has been treated with a corticosteroid prior to administration of an antisense oligomer and continues to receive the steroid therapy. In some embodiments, the steroid is glucocorticoid or prednisone.

The routes of administration described are intended only as a guide since a skilled practitioner will be able to determine readily the optimum route of administration and any dosage for any particular animal and condition. Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (e.g., adeno-associated viral vectors) (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813.

In a further embodiment, pharmaceutical compositions of the disclosure may additionally comprise a carbohydrate as provided in Han et al., Nat. Comms. 7, 10981 (2016) the entirety of which is incorporated herein by reference. In some embodiments, pharmaceutical compositions of the disclosure may comprise 5% of a hexose carbohydrate. For example, pharmaceutical composition of the disclosure may comprise 5% glucose, 5% fructose, or 5% mannose. In certain embodiments, pharmaceutical compositions of the disclosure may comprise 2.5% glucose and 2.5% fructose. In some embodiments, pharmaceutical compositions of the disclosure may comprises a carbohydrate selected from: arabinose present in an amount of 5% by volume, glucose present in an amount of 5% by volume, sorbitol present in an amount of 5% by volume, galactose present in an amount of 5% by volume, fructose present in an amount of 5% by volume, xylitol present in an amount of 5% by volume, mannose present in an amount of 5% by volume, a combination of glucose and fructose each present in an amount of 2.5% by volume, and a combination of glucose present in an amount of 5.7% by volume, fructose present in an amount of 2.86% by volume, and xylitol present in an amount of 1.4% by volume.

### IV. Kits

The disclosure also provides kits for treatment of a patient with a genetic disease (e.g., DMD) which kit comprises at least an antisense molecule (e.g., casimersen), packaged in a suitable container, together with instructions for its use. The kits may also contain peripheral reagents such as buffers, stabilizers, etc. Those of ordinary skill in the field should appreciate that applications of the above method has wide application for identifying antisense molecules suitable for use in the treatment of many other diseases.

### EXAMPLES

### Example 1: Essence

ClinicalTrials.gov Identifier: NCT02500381

The main objective of the study is to evaluate the efficacy of Casimersen (SRP-4045) and Golodirsen (SRP-4053) compared to placebo in Duchenne muscular dystrophy (DMD) patients with out-of-frame deletion mutations amenable to skipping exon 45 and exon 53, respectively.
Study Type: Interventional
Study Design: Allocation: Randomized
   Intervention Model: Parallel Assignment
   Masking: Quadruple (Participant, Care Provider, Investigator, Outcomes Assessor)
   Primary Purpose: Treatment
Official Title: A Double-Blind, Placebo-Controlled, Multi-Center Study With an Open-Label Extension to Evaluate the Efficacy and Safety of SRP-4045 and SRP-4053 in Patients with Duchenne Muscular Dystrophy

Materials and Methods

Casimersen (a/k/a SRP-4045) is a PMO of the chemical structure described herein and was supplied by Sarepta Therapeutics, Inc. The Casimersen drug product was formulated at a concentration of 50 mg/mL as a sterile, isotonic, phosphate-buffered aqueous solution that is supplied in single-use vials. The drug product was diluted with normal saline (0.9% sodium chloride injection) prior to administration via IV infusion in a clinical setting.

Patients: Eligibility

Eligible patients were 7 to 13 years of age with out-of-frame deletions of the DMD gene that are amenable skipping exon 45.

Inclusion Criteria:
Diagnosed with DMD, genotypically confirmed.
Stable dose of oral corticosteroids for at least 24 weeks.
Intact right and left biceps or 2 alternative upper muscle groups.
Mean 6MWT greater than or equal 300 meters and less than or equal to 450 meters.
Stable pulmonary and cardiac function: forced vital capacity (FVC) equal to or greater than 50% predicted and left ventricular ejection fraction (LVEF) greater than 50%.

Exclusion Criteria:
Previous treatment with SMT C1100 (BMN-195) at any time
Treatment with gene therapy at any time
Previous treatment with PRO045 or PRO053 within 24 weeks prior to Week 1
Current or previous treatment with any other experimental treatment (other than deflazacort) within 12 weeks prior to Week 1
Participation in any other DMD interventional clinical study within 12 weeks prior to Week 1
Major surgery within 3 months prior to Week 1
Presence of other clinically significant illness
Major change in physical therapy regimen within 3 months prior to Week 1.

Study Design

Patients amenable to exon 45 skipping received Casimersen (SRP-4045) intravenous (IV) infusions, at 30 mg/kg for up to 96 weeks in double-blinded period. This was followed by an open label extension period in which all patients will receive open-label active treatment of Casimersen (SRP-4045) at 30 mg/kg/week IV infusions for 48 weeks in OL period (up to Week 144 in study).
Primary Outcome Measure: Change From Baseline in the Total Distance Walked During 6-Minute Walk Test (6MWT) at Week 96 [Time Frame: Baseline and Week 96].

Secondary Outcome Measures:
Change from Baseline the Total Distance Walked During 6-Minute Walk Test (6MWT) at Week 144 (Week 48 of the OL period) [Time Frame: Baseline, Week 144].
Change from Baseline in Dystrophin Protein Levels Determined by Western Blot at Weeks 48 or 96 [Time Frame: Baseline and Weeks 48 or 96].
Change from Baseline in Dystrophin Intensity Levels Determined by Immunohistochemistry (IHC) at Weeks 48 or 96 [Time Frame: Baseline and Weeks 48 or 96].
Ability to Rise Independently From the Floor [Time Frame: Week 96, Week 144].
Time to Loss of Ambulation (LOA) [Time Frame: Baseline, Week 96, Week 144].
Change From Baseline in the North Star Ambulatory Assessment (NSAA) Total Score at Week 96 and Week 144 [Time Frame: Baseline, Week 96, Week 144]. The NSAA is a functional scale devised from the Hammersmith Scale of Motor Ability specifically for use in ambulant children with Duchenne muscular dystrophy (DMD). It consists of 17 activities graded 0 (unable to perform), 1 (performs with modifications), 2 (normal movement). The scale assesses activities required to remain functionally ambulant (example, rise from the floor), activities that can be difficult even early in the disease (e.g. standing on heels) and activities that are known to progressively deteriorate over time (stand from a chair, walk). NSAA Total Score ranges from 0 to 34, with a score of 34 implying normal function.
Change From Baseline in Forced Vital Capacity Percent (FVC %) Predicted at Week 96 and Week 144 [Time Frame: Baseline, Week 96, Week 144].

Interim Analysis

Patients amenable to exon 45 skipping were randomized to receive a once-weekly intravenous (IV) infusion of casimersen dosed at 30 mg/kg (N=27) or placebo (N=16) for 96 weeks. The interim analysis was performed on data from biopsies of the bicep muscle at baseline and on-treatment at Week 48.

Key findings from the interim analysis include:
Mean dystrophin protein (% normal dystrophin as measured by western blot) increased to 1.736% of normal compared to a mean baseline of 0.925% of normal (p<0.001).
A statistically significant difference in the mean change from baseline to week 48 in dystrophin protein was observed between the casimersen-treated arm compared to the placebo arm (p=0.009 (sensitivity analysis) p=0.004 (main method analysis)).
Of the 22 patients receiving casimersen who were tested for increased exon-skipping mRNA in an initial analysis using reverse transcription polymerase chain reaction (RT-PCR), all have displayed an increase in skipping exon 45 (p<0.001) over their baseline levels, representing a 100% response rate.
In the initial analysis, a statistically significant positive correlation between exon 45 skipping and dystrophin production was observed (Spearman rank correlation=0.635, p<0.001).
In an analysis of all 27 patients receiving casimersen who were tested for increased exon-skipping mRNA using reverse transcription polymerase chain reaction (RT-PCR), all have displayed an increase in skipping exon 45 (p<0.001) over their baseline levels, representing a 100% response rate.
In the all 27 patients analysis, a statistically significant positive correlation between exon 45 skipping and dystrophin production was observed (Spearman rank correlation=0.627, p<0.001).

The study is ongoing and remains blinded to collect additional efficacy and safety data.

### Example 2

The main objective of the study is to assess the safety and tolerability of casimersen and to evaluate the pharmacokinetics (PK) of casimersen in patients with advanced-stage DMD and confirmed mutations amenable to skipping of exon 45.

Methods

A multicenter, randomized, double-blind, placebo-controlled, dose titration, phase ½ study enrolled patients with advanced stage DMD and confirmed mutations amenable to exon 45 skipping.

During the double-blind dose titration period, patients were randomized (2:1) to receive casimersen or placebo for ≈12 weeks. Patients randomized to casimersen received 4 ascending dose levels (4, 10, 20, and 30 mg/kg) administered once weekly via intravenous (IV) infusion for ≥2 weeks per dose level. After the double-blind dose titration period, the safety and efficacy of once-weekly casimersen 30 mg/kg was evaluated in an open-label extension period for up to an additional 132 weeks.

Patients: Eligibility

Eligible patients were males aged 7-21 years with a clinical diagnosis of DMD who had a confirmed genetic mutation amenable to exon 45 skipping, had stable cardiac and pulmonary function, were on a stable dose or had not received oral corticosteroids for ≥24 weeks prior to study initiation, and were nonambulatory or unable to walk ≥300 meters on the 6-minute walk test.

Study Assessments

Safety assessments included treatment-emergent adverse events (TEAEs), clinical laboratory abnormalities, abnormalities in vital signs and physical exams, and clinically significant worsening in electrocardiograms and echocardiograms.

PK assessments included area under the concentration-time curve (AUC), total body clearance (CL), maximum observed concentration (Cₘₐₓ), terminal phase half-life (t_{1/2}), time of maximum observed concentration (tₘₐₓ), and volume of distribution at steady-state (Vₛₛ).

Data were evaluated and presented using summary and descriptive statistics.

PK parameters for casimersen were calculated using noncompartmental analysis for visits when PK measures were collected serially.

Results

Of 12 patients enrolled, 11 (91.7%) completed the study. Patients randomized to casimersen treatment were generally slightly older and had more advanced disease than those randomized to placebo (Table 1).

TABLE 1

| | Placebo | | Casimersen | | Total | |
|---|---|---|---|---|---|---|
| Parameter | (n = 4) | | (n = 8) | | (n = 12) | |
| Mean (SD) age, y | 12.0 | (2.2) | 14.4 | (3.3) | 13.6 | (3.1) |
| Race, n % | | | | | | |
| White | 4 | (100) | 6 | (75.0) | 10 | (83.3) |
| Asian | 0 | | 2 | (25.0) | 2 | (16.7) |
| Ethnicity, n (%) | | | | | | |
| Hispanic or Latino | 0 | | 1 | (12.5) | 1 | (8.3) |
| Not Hispanic or Latino | 4 | (100) | 7 | (87.5) | 11 | (91.7) |
| Mean BMIa (SD), | 21.9 | (1.2) | 25.9 | (4.8) | 24.6 | (4.3) |
| kg/m² | | | | | | |
| Mean 6MWT | 115.0 | (134.2) | 0.9 | (2.5) | 39.1 | (90.0) |
| distance^{a, b} (SD), | | | | | | |
| meters | | | | | | |
| Mean time since DMD | 91.8 | (33.7) | 136.1 | (47.9) | 121.3 | (47.4) |
| diagnosis (SD), | | | | | | |
| months | | | | | | |
| Mean duration of | 84.5 | (38.3) | 80.1 | (34.5) | 81.6 | (34.1) |
| corticosteroid use | | | | | | |
| (SD), months | | | | | | |

^{a}Baseline was defined as the last value prior to the first dose of study drug.
^{b}Patients who were not ambulatory were considered to have a 6MWT distance of 0 meters.
6MWT, 6-minute walk test; BMI, body mass index; SD, standard deviation.

The mean (SD) total time on study was 144.7 (3.45) weeks. The mean (SD) duration of time on casimersen treatment during the combined study periods was 139.6 (9.26) weeks.

Safety

All patients experienced one or more treatment emergent adverse events (TEAE) described in Table 2.

TABLE 2

| | | | | | | Casimersen | | |
|---|---|---|---|---|---|---|---|---|
| | | Casimersen | Casimersen | Casimersen | Casimersen | 30 mg/kg | | |
| | | 4 mg/kg | 10 mg/kg | 20 mg/kg | 30 mg/kg | (Week 7 to end of | | Total |
| | Placebo | (Weeks 1-2) | (Weeks 3-4) | (Weeks 5-6) | (Weeks 7-8) | double-blind period) | Total | Casimersen^{a, b} |
| Patients, n (%) | (n = 4) | (n = 8) | (n = 8) | (n = 8) | (n = 8) | (n = 8) | (N = 8) | (N = 12) |
| ≥1 TEAE | 4 (100) | 5 (62.5) | 3 (37.5) | 3 (37.5) | 4 (50.0) | 7 (87.5) | 8 (100) | 12 (100) |
| ≥1 serious TEAE | 0 | 0 | 0 | 0 | 1 (12.5) | 1 (12.5) | 1 (12.5) | 3 (25.0) |
| ≥1 TEAE | 1 (25.0) | 1 (12.5) | 0 | 0 | 0 | 1 (12.5) | 2 (25.0) | 2 (16.7) |
| related to | | | | | | | | |
| treatment | | | | | | | | |
| Total no. of | | | | | | | | |
| TEAEs, by | | | | | | | | |
| severity | | | | | | | | |
| Mild | 11 | 9 | 3 | 9 | 13 | 26 | 47 | 159 |
| Moderate | 0 | 0 | 0 | 1 | 1 | 3 | 4 | 14 |
| Severe | 0 | 0 | 0 | 0 | 2 | 2 | 2 | 2 |

^{a}TEAEs reported for casimersen-treated patients in the double-blind period are also included in summaries for the casimersen period.
^{b}No patient discontinued study drug or reduced study drug dosage because of TEAEs, and no deaths occurred during the study.

No patient discontinued study drug or reduced study drug dosage because of TEAEs. Most TEAEs were mild in severity during both the double-blind (88.7%) and open-label (90.9%) treatment periods. Procedural pain and nasopharyngitis were the most commonly reported TEAEs during the double-blind and open-label treatment periods, respectively (Tables 3 and 4).

TABLE 3
Treatment-Emergent Adverse Events Reported in ≥25% of Patients in Either
the Placebo or Total Casimersen Group During the Double-Blind Treatment Period^{a}

| | | | | | | Casimersen | |
|---|---|---|---|---|---|---|---|
| | | Casimersen | Casimersen | Casimersen | Casimersen | 30 mg/kg | |
| | | 4 mg/kg | 10 mg/kg | 20 mg/kg | 30 mg/kg | (Week 7 to end of | |
| | Placebo | (Weeks 1-2) | (Weeks 3-4) | (Weeks 5-6) | (Weeks 7-8) | double-blind period) | Total |
| Patients, n (%) | (n = 4) | (n = 8) | (n = 8) | (n = 8) | (n = 8) | (n = 8) | (N = 8) |
| ≥1 TEAE | 4 (100) | 5 (62.5) | 3 (37.5) | 3 (37.5) | 4 (50.0) | 7 (87.5) | 8 (100) |
| Procedural pain | 1 (25.0) | 0 | 0 | 2 (25.0) | 2 (25.0) | 3 (37.5) | 4 (50.0) |
| Headache | 0 | 1 (12.5) | 0 | 0 | 1 (12.5) | 2 (25.0) | 3 (37.5) |
| Vomiting | 0 | 0 | 0 | 2 (25.0) | 1 (12.5) | 2 (25.0) | 3 (37.5) |
| Nausea | 0 | 1 (12.5) | 1 (12.5) | 1 (12.5) | 0 | 0 | 2 (25.0) |
| Nasopharyngitis | 1 (25.0) | 1 (12.5) | 0 | 0 | 1 (12.5) | 1 (12.5) | 1 (12.5) |
| Pain in extremity | 1 (25.0) | 0 | 0 | 0 | 1 (12.5) | 1 (12.5) | 1 (12.5) |
| Skin papilloma | 1 (25.0) | 0 | 0 | 0 | 1 (12.5) | 1 (12.5) | 1 (12.5) |
| Dermatitis contact | 2 (50.0) | 0 | 0 | 0 | 0 | 0 | 0 |
| Back pain | 1 (25.0) | 0 | 0 | 0 | 0 | 0 | 0 |
| Ligament sprain | 1 (25.0) | 0 | 0 | 0 | 0 | 0 | 0 |
| Oropharyngeal pain | 1 (25.0) | 0 | 0 | 0 | 0 | 0 | 0 |
| Tinea versicolor | 1 (25.0) | 0 | 0 | 0 | 0 | 0 | 0 |

^{a}Only TEAEs wth an onset date after the first dose of study drug was administered until the first dose in the open-label period are included.

TABLE 4
Treatment-Emergent Adverse Events Reported in ≥25%
of Patients in the Total Casimersen Group Drug the
Double-Blind, Open-Label Treatment Period^{a}

| Patients, n (%) | Total Casimersen Group (N = 12) |
|---|---|
| ≥1 TEAE | 12 (100) |
| Nasopharyngitis | 9 (75.0) |
| Cough | 4 (33.3) |
| Headache | 4 (33.3) |
| Procedural pain | 4 (33.3) |
| Upper respiratory tract infection | 4 (33.3) |
| Vomiting | 4 (33.3) |
| Nausea | 3 (25.0) |
| Pain in extremity | 3 (25.0) |
| Oropharyngeal pain | 3 (25.0) |
| Rash | 3 (25.0) |
| Tibia fracture | 3 (25.0) |

^{a}Only TEAEs with an onset date after the first dose of casimersen was administered are included.

Treatment-related TEAEs included 1 case of moderate iron deficiency and 1 case of mild flushing in 2 casimersen-treated patients, and mild contact dermatitis in 1 patient who received placebo. Casimersen-related TEAEs resolved during the study period; the placebo-related TEAE was ongoing at the end of the study.

Five serious TEAEs (tibia fracture in 1 patient, bacteremia, septic embolus, and vena cava thrombosis in 1 patient, and femur fracture in 1 patient) occurred in patients receiving casimersen 30 mg/kg during the combined treatment periods. All 5 events were considered not related to treatment, resolved during the study, and did not recur with further dosing.

No patterns, trends, or abnormalities were observed in hematology, coagulopathy, chemistry, or other clinical laboratory parameters. No cardiac signal was noted in conduction time or functional assessment by echocardiogram. One case of transient ventricular tachycardia was reported, but the event was considered unrelated to casimersen treatment and the electrocardiogram normalized without sequelae.

Pharmacokinetics

Mean plasma casimersen concentration versus time profiles were similar at Week 7 and Week 60 for the 30 mg/kg dose level. All PK parameters were similar at Weeks 7 and 60 for the casimersen 30 mg/kg dose level (Table 5).

TABLE 5
Summary of Key Plasma Casimersen Non-compartmental
PK Parameters, by Dose Level and Week

| | Week 1 | Week 3 | Week 5 | Week 7 | Week 60 |
|---|---|---|---|---|---|
| | Casimersen | Casimersen | Casimersen | Casimersen | Casimersen |
| | 4 mg/kg | 10 mg/kg | 20 mg/kg | 30 mg/kg | 30 mg/kg |
| Parameter | (n = 8) | (n = 8)^{a} | (n = 8) | (n = 8) | (n = 12) |
| Cₘₐₓ, ng/mL | 13,700 | 39,400 | 64,400 | 119,000 | 115,000 |
| | (25.0) | (11.7) | (27.4) | (33.6) | (31.5) |
| tₘₐₓ, h | 1.1 | 1.03 | 1.03 | 0.94 | 0.95 |
| | (0.9-1.2) | (0.9-1.2) | (0.8-1.2) | (0.8-1.2) | (0.8-1.1) |
| AUC_{∞}, | 23,300 | 58,300 | 101,000 | 189,000 | 182,000 |
| h · ng/mL | (29.5) | (16.0) | (17.7) | (27.5) | (33.9) |
| Vₛₛ, L/kg | 0.369 | 0.343 | 0.407 | 0.319 | 0.367 |
| | (24.4) | (12.5) | (23.4) | (31.4) | (28.9) |
| CL, L/h/kg | 0.177 | 0.181 | 0.205 | 0.163 | 0.180 |
| | (29.1) | (15.9) | (18.5) | (27.7) | (35.0) |
| t_{1/2}, h | 2.92 | 3.29 | 3.71 | 3.82 | 3.45 |
| | (0.985) | (0.640) | (0.616) | (0.741) | (0.359) |

Values are presented as geometric mean (geometric coefficient of variation percentage) for all parameters except tₘₐₓ and t_{1/2}; tₘₐₓ is presented as median (minimum-maximum); t_{1/2} is presented as mean (standard deviation).
^{a}At Week 3, n = 7 for AUC_{∞}, Vₛₛ, CL, and t_{1/2}.
AUC_{∞}, area under the concentration-time curve from time 0 extrapolated to infinity.

### SUMMARY

In this study, Casimersen 30 mg/kg was well tolerated in patients with advanced-stage DMD and confirmed mutations amenable to exon 45 skipping. Most reported TEAEs were mild in severity, few treatment related TEAEs were reported, and no patients discontinued study drug or reduced study drug dosage because of TEAEs. No clinically significant laboratory abnormalities or worsening in electrocardiograms and echocardiograms was noted. Casimersen PK analyses suggest little to no accumulation following weekly dosing at 30 mg/kg.

All publications and patent applications cited in this specification are herein incorporated by reference as if each individual publication or patent application were specifically and individually indicated to be incorporated by reference.

1. A method for treating Duchenne muscular dystrophy (DMD) in a patient in need thereof who has a mutation of the DMD gene that is amenable to exon 45 skipping, comprising administering to the patient a dose of casimersen or a pharmaceutically acceptable salt thereof.

2. The method according to claim 1, wherein the dose is administered at a dosage of about 30 mg/kg of body weight of the patient.

3. The method according to claims 1-2, wherein the dose is administered as a single dose.

4. The method according to claims 1-3, wherein the dose is administered once weekly.

5. The method according to any of the previous claims, wherein the patient has a mutation of the DMD gene that is selected from the group consisting of: exons 7 to 42, 12 to 42, 18 to 42, 44 to 46, 44 to 47, 44 to 48, 44 to 49, 44 to 51, 44 to 53, 44 to 55, 44 to 57, or 44 to 59, or exon 44.

6. The method according to any of the previous claims, wherein the patient is chronically administered casimersen.

7. The method according to any of the previous claims, wherein the patient is administered casimersen for at least 48 weeks.

8. The method according to any of the previous claims, wherein the patient is on a stable dose of corticosteroids for at least 6 months prior to administration of casimersen.

9. The method according to any of the previous claims, wherein casimersen or a pharmaceutically acceptable salt thereof is formulated as a pharmaceutical composition.

10. The method according to any of the previous claims, wherein casimersen or a pharmaceutically acceptable salt thereof is formulated as a pharmaceutical composition having a strength of about 50 mg/mL.

11. The method according to claim 10, wherein casimersen or a pharmaceutically acceptable salt thereof is formulated as a pharmaceutical composition having a strength of about 50 mg/mL and presented in a dosage form of about 100 mg/2 mL.

12. The method according to claim 11, wherein the dosage form is contained in a single-use vial.

13. The method according to claims 10-12, wherein casimersen or a pharmaceutically acceptable salt thereof is formulated as a pharmaceutical composition comprising casimersen or a pharmaceutically acceptable salt thereof and a pharmaceutically acceptable carrier.

14. The method according to claim 13, wherein the pharmaceutically acceptable carrier is a phosphate-buffered solution.

15. A method for restoring an mRNA reading frame to induce exon skipping in a patient with Duchenne muscular dystrophy (DMD) in need thereof who has a mutation of the DMD gene that is amenable to exon 45 skipping, comprising administering to the patient a dose of casimersen or a pharmaceutically acceptable salt thereof.

16. The method according to claim 15, wherein the dose is administered at a dosage of about 30 mg/kg of body weight of the patient.

17. The method according to claims 15-16, wherein the dose is administered once weekly.

18. The method according to according to claims 15-17, wherein the patient is administered casimersen for at least 48 weeks.

19. A method for increasing dystrophin production in a patient with Duchenne muscular dystrophy (DMD) in need thereof who has a mutation of the DMD gene that is amenable to exon 45 skipping, comprising administering to the patient a dose of casimersen or a pharmaceutically acceptable salt thereof.

20. The method according to claim 19, wherein the dose is administered at a dosage of about 30 mg/kg of body weight of the patient.

21. The method according to claims 19-20, wherein the dose is administered once weekly.

22. The method according to claims 19-21, wherein the patient is administered casimersen for at least 48 weeks.

23. The method of any of the previous claims, further comprising confirming that the patient has a mutation in the DMD gene that is amenable to exon 45 skipping prior to administering casimersen.

24. Casimersen or a pharmaceutically acceptable salt thereof for use in treating Duchenne muscular dystrophy (DMD) in a patient in need thereof, the patient having a mutation of the DMD gene that is amenable to exon 45 skipping, wherein the treatment comprises administering to the patient a single intravenous dose of casimersen of about 30 mg/kg once weekly.

25. Casimersen or a pharmaceutically acceptable salt thereof for use in restoring an mRNA reading frame to induce exon skipping in a patient with Duchenne muscular dystrophy (DMD) in need thereof, the patient having a mutation of the DMD gene that is amenable to exon 45 skipping, wherein the treatment comprises administering to the patient a single intravenous dose of casimersen of about 30 mg/kg once weekly.

26. Casimersen or a pharmaceutically acceptable salt thereof for use in increasing dystrophin production in a patient with Duchenne muscular dystrophy (DMD) in need thereof, the patient having a mutation of the DMD gene that is amenable to exon 45 skipping, wherein the treatment comprises administering to the patient a single intravenous dose of casimersen of about 30 mg/kg once weekly.
